(19) United States
(12) Patent Application Publication
(10) Pub. No.: US 2019/0057773 A1
(43) Pub. Date: Feb. 21, 2019

(54) METHOD AND SYSTEM FOR PERFORMING TRIAGE

(71) Applicant: BOE TECHNOLOGY GROUP CO., LTD., Beijing (CN)

(72) Inventor: Hui LI, Beijing (CN)

(21) Appl. No.: 15/993,522

(22) Filed: May 30, 2018

(30) Foreign Application Priority Data
Aug. 15, 2017 (CN) 201710697575.7

(51) Int. Cl. G16H 50/20; G06F 17/30; G06N 5/02

(52) U.S. Cl. CPC G16H 50/20; G06N 5/02; G06F 17/30654

(57) ABSTRACT

This disclosure relates to a method and system for performing triage. The method includes obtaining a descriptive statement inputted by a user; analyzing the descriptive statement to extract predefined class information, the predefined class information being disease related information that belongs to a predefined class; querying, according to the predefined class information, to obtain the triage information when the query condition for querying triage information is met based on the predefined class information, and informing the user of the triage information. This enables a triage process to be intelligent, which reduces operational burdens on both sides of the medical institution and the user.

### RELATED APPLICATION

The present application claims the priority of the Chinese patent application No. 201710697575.7 filed on Aug. 15, 2017, the entire disclosure of which is incorporated herein by reference.

### FIELD

This disclosure relates to a method and system for performing triage.

### BACKGROUND

Nowadays, in the field of medical treatment, a phenomenon occurring frequently is that, because patients are short of medical and health knowledge, they do not know which medical department to visit, or they would go to large hospitals for medical treatment even if they have a minor illness. This significantly reduces medical efficiency and wastes medical resources.

In order to solve such problems, remote manual triage table and applications that provide users with self-triage services have been proposed, which can provide patients with consulting service, including personalized health information, for the patients. The triage is generally directed at consultation before a hospital visit, which mainly solves the user's requirement from a phase of “malaise” to a phase of “visiting a hospital”, serves as a family doctor, and gives guidance to diagnosis and treatment.

### SUMMARY

In an aspect, an embodiment of this disclosure provides a triage method implemented by a computing device. The triage method comprises: obtaining a descriptive statement inputted by a user via the computing device; analyzing the descriptive statement to extract predefined class information, the predefined class information being disease related information that belongs to a predefined class; and querying, according to the predefined class information, to obtain the triage information when the query condition for querying triage information is met based on the predefined class information, and informing the user of the triage information.

In some embodiments, if it fails to extract the predefined class information from the obtained descriptive statement, or if it is determined based on the predefined class information that the query condition is not met, an inquiry statement is generated and displayed, so as to obtain a next descriptive statement inputted by the user.

In some embodiments, the analyzing the descriptive statement to extract predefined class information comprises: recognizing a named entity in the currently obtained descriptive statement through a pre-trained named entity recognition model. Optionally, the recognized named entity is extracted as the predefined class information when the recognized named entity is a predefined class named entity.

In some embodiments, the query condition includes at least one of the following: the extracted predefined class information includes particular class information; the extracted predefined class information includes a predetermined amount of particular class information; and the extracted predefined class information includes a piece of particular class information and the piece of particular class information is not included in a set of common predefined class information corresponding to a plurality of diseases.

In some embodiments, querying to obtain the triage information comprises: classifying each of pieces of extracted predefined class information through a pre-trained classification model; querying, at least according to the pieces of extracted predefined class information and their respective classes, to obtain the triage information.

In some embodiments, querying to obtain the triage information comprises: querying for triage information according to a pre-constructed decision tree at least based on symptom information in the extracted predefined class information.

In some embodiment, informing the user of the triage information comprises displaying the triage information on a user interface of the computing device. When it is determined based on the predefined class information that the query condition is not met, the user interface is jumped to a remote manual diagnosis interface if the number of times of obtaining the descriptive statement exceeds a threshold.

In some embodiments, the method further comprises: generating and displaying a statement that inquires whether to register or not after the triage information has been given; and determining whether to provide a link associated with the triage information based on response information inputted by the user.

In some embodiments, the descriptive statement is analyzed through natural language processing (NLP).

In some embodiments, the predefined class information includes any one or more of symptom class information, physiological parameter class information, time class information, basic feature class information, and medical history class information.

In some embodiments, the method further comprises calculating a total weighted value of the extracted predefined class information after the extracting the predefined class information, and determining that the query condition is met when the weighted value exceeds a predefined threshold.

In some embodiments, the triage information includes at least one of disease information and corresponding department information.

In another aspect, an embodiment of this disclosure provides a system for triage implemented by a computing device. The triage system comprises: an interface, a statement analyzer and a querier. The interface is configured to obtain a descriptive statement inputted by a user. The statement analyzer is configured to analyze the descriptive statement to extract predefined class information. The predefined class information is disease related information that belongs to a predefined class. The querier is configured to query, according to the predefined class information, to obtain the triage information, when a query condition for querying triage information is met based on the predefined class information. The interface is further configured to inform the user of the triage information.

In some embodiments, the query condition includes at least one of the following: the extracted predefined class information includes particular class information; the extracted predefined class information includes a predetermined amount of particular class information; and the extracted predefined class information includes a piece of particular class information and the piece of particular class information is not included in a set of common predefined class information corresponding to a plurality of diseases.

In some embodiments, the system further comprises an inquiry module. The inquiry module is configured to generate an inquiry statement when the statement analyzer fails to extract the predefined class information from the currently obtained descriptive statement, or when the querier determines that the query condition is not met. The interface is further configured to display the inquiry statement so as to obtain a next descriptive statement inputted by the user.

In some embodiments, the system further comprises a remote manual diagnosis module. The remote manual diagnosis module is configured to obtain the descriptive statement through the interface so as to perform manual triage, and send remote manual triage information to the interface for display. The querier is further configured to, when it is determined based on the predefined class information that the query condition is not met, instruct the interface to establish a connection with the remote manual diagnosis module to perform manual triage when the number of times of obtaining the descriptive statement exceeds a threshold.

In some embodiments, the system further comprises a registration module. The registration module is configured to generate a link associated with the triage information. The interface is further configured to cause the link to be displayed to the user.

In a further aspect, an embodiment of this disclosure provides a computing device. The computing device comprises a memory, a processor and a computer program stored on the memory and running on the processor. The processor, when executing the program, performs the steps of the method stated previously and in other parts of this disclosure.

In yet another aspect, an embodiment of this disclosure provides a computer readable storage medium. The computer readable storage medium stores a computer readable instruction. The computer readable instruction, when being executed by the processor, implements the steps of the method stated previously and in other parts of this disclosure.

### BRIEF DESCRIPTION OF THE DRAWINGS

This disclosure will be better understood by reference to the following drawings and in conjunction with the accompanying specification.

FIG. 1 is a schematic flow chart of a method for triage according to an embodiment of this disclosure;

FIG. 2 is a schematic flow chart of a method for triage according to another embodiment of this disclosure;

FIG. 3 is a schematic view of a user interface displayed on a screen according to an embodiment of this disclosure; and

FIG. 4 is a schematic block diagram of a system for triage according to an embodiment of this disclosure.

### DETAILED DESCRIPTION

Embodiments of the present disclosure are now described in detail below with reference to the drawings. In the following description, for purposes of explanation, numerous specific details are set forth in order to provide an understanding of the claimed subject matter. It may be evident, however, that the claimed subject matter may be practiced without these specific details. In other instances, structures and devices are illustrated in block diagram form in order to facilitate describing the claimed subject matter. Further, in this context, and for the purpose of brevity and clarity, detailed descriptions of well-known apparatus, circuits and methodology have been omitted so as to avoid unnecessary detail and possible confusion.

Systems for triage in relevant art generally provide users with hierarchical interfaces for selecting diseases. As such, a user is required to make manual selection of options on a respective interface of each hierarchy based on his/her own disease to achieve the triage. Because users have to be familiar with options on interfaces of all of hierarchies and must traverse interfaces of every hierarchy during use of these triage systems, such a self-triage process is relatively mechanical for the users and wastes their time and energy.

To this end, the embodiments of the present disclosures provide a method and system for performing triage.

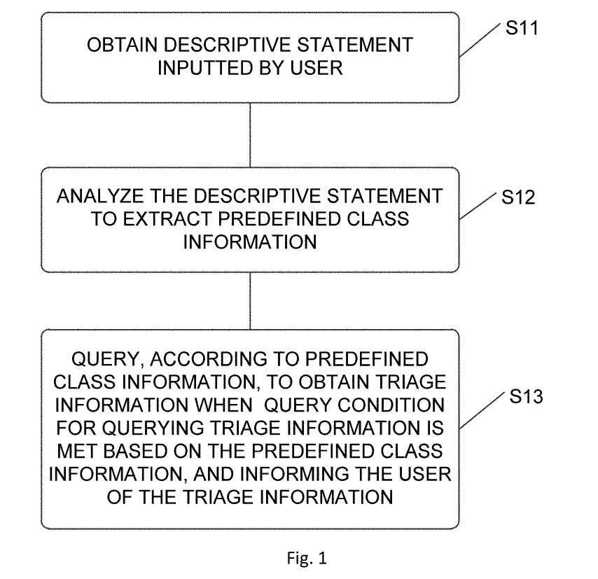

FIG. 1 is a schematic flow chart of an intelligent triage method according to an embodiment of this disclosure. The method may be performed in connection with any suitable hardware, software, firmware, or combination thereof. In some embodiment, the method may be performed by a computing device. In at least some embodiments, the method may be performed by software in the form of computer readable instructions, embodied on some type of computer-readable storage medium, which may be performed under the influence of one or more processors.

As shown in FIG. 1, the intelligent triage method according to an embodiment of this disclosure comprises the following steps.

At S11, a descriptive statement inputted by a user is obtained via the computing device.

In some embodiments, the descriptive statement inputted by the user using an input device such as keyboard, mouse, touch screen or microphone may be received via a user interface. The user interface may be for example a man-machine dialogue interface provided in the form of a web page window on the computing device, or a man-machine dialogue interface provided through applications installed on the computing device. The computing device for example may be a desktop, a portable, a smart phone, a tablet and the like. In one configuration, the computing device may comprise a memory and at least one processor. The computer program may be stored in the memory so as to be executed by the processor.

In one example, after a dialogue interface is presented as a man-machine interface, the descriptive statement inputted by the user may be obtained through a dialogue box in the dialogue interface.

The descriptive statement may be natural language text. The descriptive statement may be a complete sentence, such as “I began to be dizzy yesterday” or “I was bitten by a poisonous snake”, and may also be one or more discrete phrases or figures, such as “high pressure 180, low pressure 110” or simply “180/110”.

The descriptive statement may include statements describing a specific symptom of current malaise of the patient. In some embodiments, the descriptive statement may also include statements describing the patient's physiological parameters such as blood pressure, blood sugar and/or statements describing time information such as the duration or starting time of the current abnormal state of his/her body. Alternatively or additionally, the descriptive statement may also include statements describing the patient's basic features such as height, weight, age and/or statements describing medical history such as family medical history, personal medical history.

In some embodiments, the man-machine dialogue in the dialogue box may start from the service side (e.g., a service provider of triage service) and may also start from the client side (e.g., the user of the triage service). For example, the service provider may provide guidance statements in the dialogue box first, so as to let the user provide effective information for the triage faster and more targeted, thereby providing a better experience for the user.

At S12, statement analysis is performed on the obtained descriptive statement to extract predefined class information. The predefined class information is disease related information that belongs to a predefined class.

According to an embodiment of this disclosure, natural language processing (NLP) may be used for performing statement analysis. In some embodiments, the natural language processing may be performed based on medical corpus. The medical corpus collects medical related corpus, including but not limited to disease related sentences, phrases, words and so on. In some embodiments, the medical corpus may collect more medical related corpora through machine learning. The medical corpus may be located on the computing device locally or may be located remotely away from the computing device. In one embodiment, the medical corpus is a “cloud” server farm, which includes one or more server computers that are connected to the computing device through a network or the Internet or other means.

In some embodiments, for the obtained descriptive statement, e.g. the statement inputted into the dialogue box, recognition of e.g., words or phrases or sentences may be performed based on the medical corpus. In one example, the descriptive statement may be fragmented into a plurality of possible word units according to a preset word count. By comparing each word unit with word or phrase or sentence entries in the medical corpus, the words or phrases or sentences in the descriptive statement matching with them are recognized, and thus disease related useful information is extracted. In another example, a statement recognition model may also be trained based on corpus data in the medical corpus, and the trained statement recognition module is used to recognize the related words or phrases or even sentences in the descriptive statement, and thus extracting useful disease related information.

In some embodiments, the disease related information is classified into different classes according to certain rules. For example, the predefined classes may include symptom class, physiological parameter class, time class, basic feature class, medical history class and so on. It could be understood that such classification is exemplary, and other suitable classification may also be adopted with the continuous development of the medical technology.

Accordingly, the medical corpora may also be classified into predefined classes.

In some embodiments, the extracted information may be determined to be predefined class information by determining to which predefined class(es) in the medical corpus the words or phrases or sentences corresponding to those recognized from the descriptive statement belong. For example, the predefined class information may include but not limited to symptom class information, physiological parameter class information, time class information, basic feature class information, medical history class information and so on.

In some embodiments, when the descriptive statement does not contain any disease related information, for example, when no corpus matching therewith is found in the medical corpus through statement analysis, information extraction fails. At this point, guidance prompt may be given to the user, including e.g., example descriptive statement, so that the user may input useful information.

At S13, query is performed, according to the predefined class information, to obtain the triage information when the query condition for querying triage information is met based on the predefined class information.

In some embodiments, whether a query condition(s) for querying triage information is met is determined based on the predefined class information.

The query may be performed based on a medical knowledge base. The medical knowledge base is medical related knowledge base established based on expert systems. In one embodiment, the medical knowledge base may be located on the computing device locally or may be located remotely away from the computing device. In one embodiment, the medical knowledge base is a “cloud” server farm, which includes one or more server computers connected to the computing device through a network or the Internet or other means.

In some embodiments, the query condition(s) is set up, so that whether the extracted predefined class information is sufficient to query significant/effective triage information is determined before querying about triage. As an example, a query condition may be that the query may be performed only if the extracted predefined class information includes a particular class of information such as symptom class information. As another example, a query condition may be that the query may be performed only if the extracted predefined class information includes a predetermined amount of particular class information, e.g., including more than two pieces of symptom class information. As a further example, when the predefined class information only includes one particular class of information, e.g., the symptom class information, a query condition may be that the query may be performed only if the particular class of information is not included in a set of particular common class information corresponding to a plurality of diseases. For example, it is assumed that the extracted predefined class information only includes one piece of symptom class information like “dizzy” or “fever”. Because this symptom is likely to occur for a variety of diseases, this symptom is included in a set of common symptom class information shared by a plurality of diseases. If a query is performed based on such symptom class information, the query result will include a relatively large amount of disease information. Because such a query result may not play an effective function in providing references for triage, it may be determined that such predefined class information does not meet the query condition.

In some embodiments, different weights may be assigned to different classes of information. After the predefined class information is extracted, a total weighted value of the extracted predefined class information may be calculated. When the weighted value exceeds a predefined threshold, it is determined that the query condition is met.

In some embodiments, query is performed to obtain the triage information based on the predefined class information if the query condition is met, and the user is informed of the triage information.

In some embodiments, when the query condition is determined to be met and if the extracted predefined class information contains a plurality of pieces of information belonging to the same class or different classes, these predefined class information may be combined to query comprehensively the disease information that matches with the combined information best. For example, a group(s) of predefined class information related to a disease may be created in advance in the medical knowledge base. For each disease, a corresponding correlation value is assigned to each of its related predefined classes. When performing query, the query may be performed on the group(s) of information corresponding to the disease according to individual pieces of predefined class information recognized from the descriptive statement. When the query result indicates that a plurality of diseases are involved, a correlation between the combined predefined class information and one of the diseases may be calculated based on respective values of correlations between every piece of predefined class information and the disease, so as to determine the probability of the combined predefined class information identifying this disease. One or more diseases with the highest probability are selected to determine the triage information accordingly.

The triage information may include disease information (including for example, disease grading information, such as acute and critical disease, acute and severe disease, less urgent, non-urgent etc.) or corresponding department information or combination thereof. After the triage information is determined, the corresponding disease information and/or the corresponding department information or the combination thereof may be displayed visually via the dialogue box. It could be understood that the triage information may also be communicated to the user audibly or in other applicable manners.

The intelligent triage method according to embodiments of this disclosure allows the user to provide disease related information in a form of natural dialogue, without traversing multi-hierarchy interfaces comprising options in a general triage system to select the symptom information, such that the user is allowed to obtain the desired triage information easily and conveniently. This reduces the operation burden at the user side greatly. Meanwhile, by setting up the query conditions, the possibility of obtaining effective triage result through querying is increased significantly; meanwhile the triage result is also refined to match the patient's disease more precisely. This saves the cost of the medical treatment of the patient in both time and expense, increases the medical efficiency, and avoids waste of the medical resources.

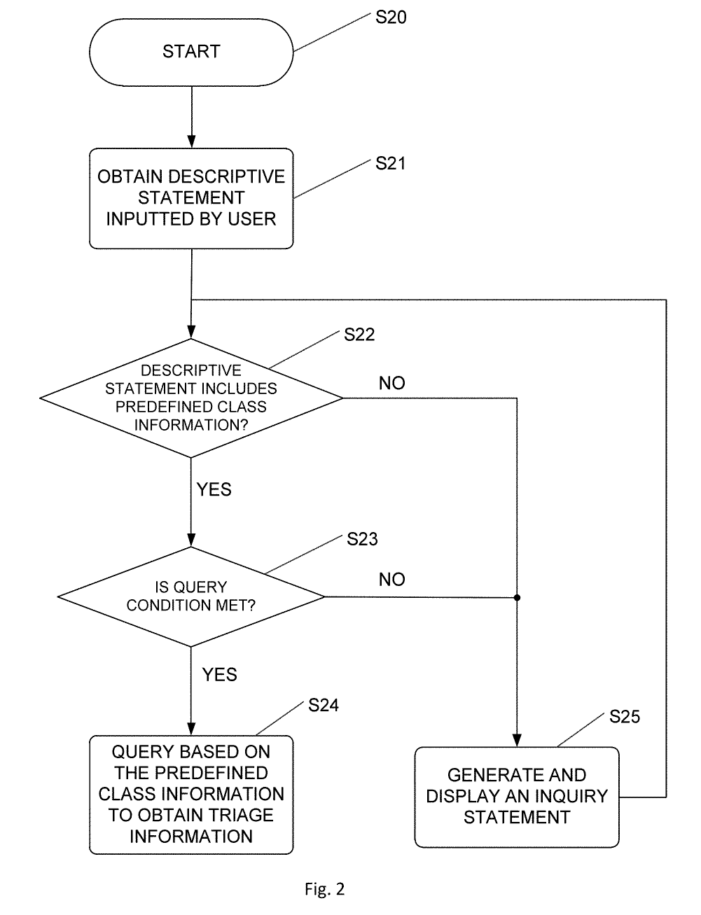

FIG. 2 is a schematic flow chart of a triage method according to another embodiment of this disclosure. The method may be carried out by combining any appropriate hardware, software, firmware or combination thereof. In some embodiments, the method may be carried out by a computing device. In at least some embodiments, the method may be carried out by software in the form of computer readable instructions stored on a certain type of computer readable storage medium. The software may be executed under the influence of one or more processors.

As shown in FIG. 2, the intelligent triage method according to an embodiment of this disclosure comprises the following steps.

At S20, the method starts. In one example, prompt information may be given to a user through a user interface to start a triage process. Alternatively, it may also simply wait for user input.

At S21, a descriptive statement inputted by the user is obtained.

The descriptive statement may include various patient related information, e.g., information that belongs to different predefined classes, as described previously, including but not limited to basic information such as height, weight, age, medical history information such as family medical history, personal medical history, specific status information of current malaise, physiological parameter information such as blood pressure, blood sugar, duration information or start time information of the current abnormal state of the body, etc.

At S22, whether currently obtained descriptive statement includes predefined class information is determined. If yes, the method proceeds to S23, otherwise, proceeds to S25.

For example, whether the descriptive statement includes the predefined class information may be determined by determining to which predefined class(es) in the medical corpus the words or phrases corresponding to those recognized from the descriptive statement belong. For example, phrases like “high pressure 180” and “low pressure 110” may be recognized from the descriptive statement, and then be determined as belong to the physiological parameter class information.

In some embodiments, different prompt information may be given to the user on the user interface according to a predesigned mode, in order to obtain user information of interest. The user may input a descriptive statement that provides corresponding information based on the prompt information.

In some embodiments, when it is determined that the descriptive statement does not contain symptom class or physiological parameter class information, prompt as to inputting corresponding information is given to the user.

At S23, the predefined class information is extracted and whether query condition is met is determined on the basis of this. If yes, the method proceeds to S24, otherwise, proceeds to S25.

In some embodiments, the query condition is set to determine, before querying, whether the extracted/collected predefined class information is sufficient to obtain effective triage information from the query. For example, the query conditions may be that whether the predefined class information currently extracted from the descriptive statement includes a predetermined amount of symptom information and a predetermined amount of auxiliary information. For example, when the extracted predefined class information contains two pieces of physiological parameter class information “high pressure 180” and “low pressure 110” simultaneously and four pieces of basic feature class information “male”, “65 years old”, “170 cm”, “70 kg”, it may be determined that the query condition is met.

At S24, query is performed based on the extracted predefined class information to obtain the triage information, and the triage information obtained from the query is displayed.

When it is determined that the query condition is met, a single piece of predefined class information may be used every time to query the medical knowledge base. For example, the information “high pressure 180” may be used for querying. Here, a query result may be derived that the blood pressure is too high and the disease might be hypertension. Alternatively, the predefined class information may be combined reasonably and the combined information may be used to query the medical knowledge base. For example, the extracted information may be combined as “male, 65 years old, 170 cm, 70 kg, high pressure 180, low pressure 110”, and this combined information is used to perform query. In this way, when determining the disease, the influence of factors such as the gender and the age of the patient may also be considered in addition to the intuitive blood pressure parameter, thus achieving a diagnosis result that matches with the patient's current situation better.

The triage information may include disease information or the corresponding department information or the combination thereof. The triage information displayed to the user may include the recommended department, and related disease information, e.g., pre-diagnosis suggestion, related medical reference information and other suggestions etc.

At S25, if it is determined that the descriptive statement does not include any predefined class information or does not meet the query conditions, an inquiry statement is generated and displayed, so that a next descriptive statement inputted by the user may be obtained.

The descriptive statement obtained in S21 may possibly not include predefined class information. For example, some possible cases may be that the user inputs some descriptive statements unrelated to the triage in the dialogue box, e.g., the user may input statements like “hello”, “what service do you provide?”, “I want to ask whether my relative should go to see the doctor in that situation”, either intentionally or unintentionally, when he sees the greetings shown in the dialogue box. Such statements do not include any information that helps to perform triage judgement. In such a case, the inquiry statement may be displayed in the dialogue box to guide the user to input information that is helpful for triage judgement. The inquiry statement for example may be “may I have your gender, age, height and weight?” Additionally or alternatively, the inquiry statement may also be “do you feel chest tightness or shortness of breath?”, “is there any other symptom?” or any other applicable statements.

In addition, all of the predefined class information extracted from the descriptive statement in S23 possibly fails to meet the query conditions. For example, the user may only input such descriptive statement like “I feel dizzy”. Because only one piece of symptom class information “dizzy” may be extracted from this descriptive statement, it may not meet the query condition that for example requires containing more than two pieces of symptom class information. Here, similar inquiry statement may also be applied to obtain more predefined class information.

The intelligent triage method according to an embodiment of this disclosure may send an inquiry statement to the user automatically when the information required by triage query is insufficient, to guide the user to provide more descriptive statements that help to perform triage judgement. This enables the triage process to be further intelligent, and may provide service that is similar as the manual triage for the user.

In some embodiments, the statement analysis on the descriptive statement may be performed in various ways. In one example, the predefined class information may be extracted from the descriptive statement by e.g., recognizing a named entity in the currently obtained descriptive statement through a pre-trained named entity recognition model. It is confirmed whether the recognized named entity includes a predefined class named entity. If yes, the predefined class named entity is determined as the predefined class information.

In one example, the named entity is recognized in the inputted descriptive statement, and then the inputted descriptive statement is classified based on the predefined type. The named entity recognition model may be implemented using for example the Stanford Named Entity Recognizer. Specifically, a named entity recognition model for extracting predefined class information from the descriptive statement may be constructed by training a named entity recognizer using certain scale of training corpus prepared in advance in the medical corpus, e.g., medical related tagged data. The trained named entity recognition model may be used for locating the disease related named entity in the descriptive statement and classify it into a predefined class.

The named entity in an embodiment of this disclosure for example may include but not limited to symptom class named entity, time class named entity, physiological parameter class named entity, basic feature class named entity, medical history class named entity etc. For example, the symptom class named entity may include words mainly for determining the triage information such as “dizzy”, “fever”, “diarrhea”, “heatstroke”. The time class named entity for example may include auxiliary words for determining the triage information such as “yestermorning”, “two days”, “today”. The physiological parameter class named entity for example may include words for directly or indirectly determining the triage information such as “high pressure/low pressure”, “blood sugar”. The physiological parameter class named entity may also be determined as corresponding predefined class information together with the digital information adjacent with this type of named entity. The basic feature class named entity for example may include information like “year of age”, “cm”, “kg” representing the age, height and weight. Similarly, the basic feature class named entity may be determined as corresponding predefined class information together with the digital information adjacent with this type of named entity.

In some embodiments, for recognition of the named entity, for example, recognition of time or symptom or any defined type, a sequence labelling algorithm in a machine learning algorithm, e.g., conditional random field algorithm may be utilized. A label, e.g., BIO, may be predefined in the sequence labelling algorithm, wherein ‘B’ represents the beginning of a phrase representative of time (such as “yesterday” of “yesterday evening”), ‘I’ represents a word of the phrase representative of time not at the beginning (such as “evening” of “yesterday evening”), and ‘O’ represents a word that does not belong to the phrase representative of time. The conditional random field algorithm is a supervise algorithm, which may tag the training corpus and obtain a trained model via learning for use in automatic prediction.

In an embodiment of this disclosure, the training corpus used by the named entity recognition model in training, in addition to some general named entity expressions, may also include some related named entity expressions. For example, for the written expression of “diarrhea”, considering that it is natural language man-machine dialogue in the dialogue box, some colloquial expressions may also be taken as the training corpus to train the named entity recognition model. For example, the colloquial expressions “have loose bowels”, “have got a run” associated with “diarrhea” are taken as the training corpus. As another example, for the time class named entity, the conventional named entity may include “tomorrow”, “yesterday”, “today” etc. Meanwhile, some colloquial expressions such as “tmr”, “ystd”, “2dae” may also be taken as the time class training corpus to train the named entity recognition model.

In another embodiment of this disclosure, querying to obtain the triage information for example may include classifying the extracted predefined class information to obtain the corresponding class information through a pre-trained classification model, and performing query based on the extracted predefined class information and its corresponding class information to obtain the triage information. In an embodiment of this disclosure, the extracted predefined class information may be classified into different classes according to intentions. Some intention-related classes may be predefined, e.g., “emergency class” and “non-emergency class”. In one example, certain training corpus may be tagged to be of different intention classifications when training a classifier, for example, tagging “cough” as “non-emergency class” while tagging “hemorrhoea” as “emergency class”. The tagged training corpus may be utilized to train the classifier using a support vector machine (SVM). The trained classifier may classify the descriptive statement inputted by the user so as to determine whether it is emergent, and give corresponding triage information, e.g., disease grading information.

In an embodiment of this disclosure, querying to obtain the triage information may be implemented as, based on the extracted predefined class information, querying corresponding triage information from a pre-constructed decision tree. For example, related disease may be queried from the decision tree pre-constructed by experts through a depth first search strategy based on the symptom class information. The decision tree for example is a binary tree, each edge thereof representing a symptom condition. For example, with respect to the symptom of cough, the left subtree corresponds to cough a, and the right subtree corresponds to no cough b; while with respect to the symptom of fever, the left subtree corresponds to fever c, and the right subtree correspond to no fever d. When the symptom information determined in all of the descriptive statements obtained from the dialogue box during the current dialogue process is “cough, no fever . . . ” the corresponding process of querying the decision tree for example includes a→d→ . . . . When the determined symptom information is “cough, fever . . . ” the corresponding process of querying the decision tree for example includes a→c→ . . . . By querying the pre-constructed decision tree, a leaf nod that corresponds to a disease may be reached by starting from the root node of the corresponding decision tree and stepping down to the leaf node based on the determined symptom information.

In embodiments of this disclosure, determination as to whether the query condition is met may be further implemented in such a way that, after starting to query the decision tree based on a predetermined amount of the currently obtained symptom information, every time a symptom node is traversed, whether the obtained symptom information includes symptom information corresponding to that of the symptom node is determined. If yes, the process proceeds to a next symptom node. Otherwise, an inquiry statement for inquiring about a symptom(s) at the symptom node may be generated and entered into the dialogue box. Alternatively, after starting to query the decision tree based on a predetermined amount of the currently obtained symptom information, every time a symptom node is traversed, whether the obtained symptom information includes symptom information corresponding to that of the symptom node is determined. If yes, a next symptom node is traversed as a consequence. Otherwise, it may be deemed by default that there is no symptom information corresponding to that of the symptom node, thereby the process proceeding to a next symptom node.

In the embodiments of this disclosure, if it is determined based on the predefined class information extracted from the descriptive statement that the query condition is not met, a step of determining whether the number of times of obtaining descriptive statements from the dialogue box exceeds a threshold may be further performed. In an embodiment of this disclosure, for some disease status that may not be determined from the medical knowledge base, or for some cases that no effective symptom information is given in response to the inquiry statement, a threshold may be set for the number of times of query, e.g., 10. If the triage information still cannot be determined after the number of times of dialogues exceeds the threshold, the current dialogue interface may be jumped to a remote manual diagnosis interface, where the manual triage service may be provided by remote professionals.

Additionally, after the triage information is displayed, a prompt for registration may be given to the user by, for example, generating a statement of asking whether to register and displaying the statement in the dialogue box. A link associated with the triage information may be provided as desired by the user.

Various operations of embodiments are provided herein. In one embodiment, one or more of the described operations may constitute computer readable instructions stored on one or more computer readable mediums, which if executed by a computing device, will cause the computing device to perform the described operations. The order in which some or all of operations are described should not be interpreted as implying that these operations are necessarily order dependent. The skilled person in the art having the benefit of this specification will appreciate alternative ordering. In addition, it would be understood that not all operations are necessarily present in each embodiment provided herein.

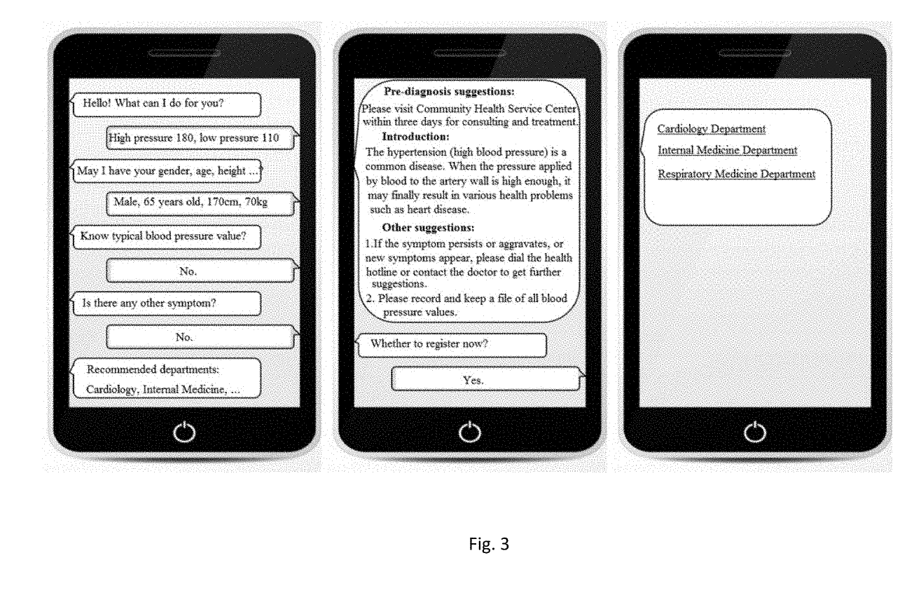

FIG. 3 is a schematic user interface for communicating in natural language between a human being and a computing device to perform intelligent triage according to an embodiment of this disclosure.

As shown in the user interface at the left of FIG. 3, the greetings: “hello! What can I help your?” may be displayed in a dialogue box. A descriptive statement: “high pressure 180, low pressure 110” inputted by the user is then obtained. The phrases “high pressure 180” and “low pressure 110” may be recognized from this descriptive statement, and thus physiological parameter class information may be extracted accordingly.

Afterwards, if it is desired to obtain more user information, a prompt like “may I have your gender, age, height and weight” may be given. Here, the user further inputs a descriptive statement of “male, 65 years old, 170 cm, 70 kg”. Words, such as “male”, “65 years old”, “170 cm”, “70 kg”, may be recognized from this descriptive statement, and thus such information may be determined as belonging to the basic feature class information accordingly.

When it is determined that a query condition(s) is met based on these predefined class information, they may be used to query a medical knowledge base. A triage result obtained for example after querying a decision tree based on the predefined class information determined from the descriptive statements is shown in the user interfaces at the left and the middle of FIG. 3. The suggested departments for medical treatment are: Internal Medicine-Cardiovascular Department, General Internal Medicine Department and Respiratory Medicine Department. Meanwhile, pre-diagnosis suggestions and reference information as well as other suggestions have also been given.

Additionally, in some embodiments, information on medical treatment may also be given. As shown in the user interface at the middle of FIG. 3, an inquiry statement of “whether to register now?” may also be given in the dialogue box. When the response message inputted by the user in the dialogue box is “yes”, links associated with the three triage departments as shown in the user interface at the right of FIG. 3 are given. The user may jump the current interface to the registration interface to register or make an appointment by clicking the link directly.

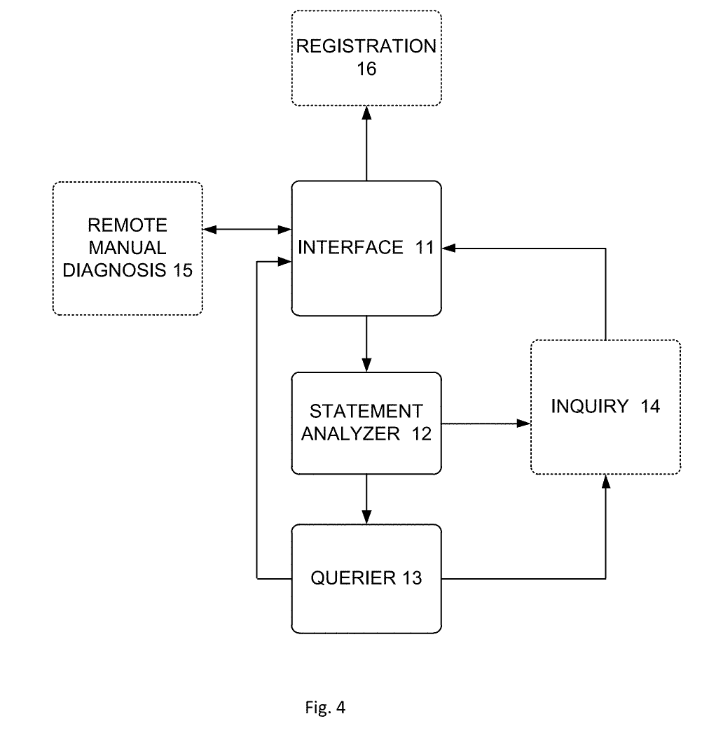

FIG. 4 is a schematic block diagram of an intelligent triage system according to an embodiment of this disclosure. The intelligent triage system may be used for carrying out the triage method according to embodiments of this disclosure.

As shown in FIG. 4, the intelligent triage system according to an embodiment of this disclosure may comprise an interface 11, a statement analyzer 12 and a querier 13.

The interface 11 is configured to obtain a descriptive statement inputted by a user. The interface may receive the descriptive statement inputted by the user e.g. through a man-machine interaction interface in the form of a web page window on a computing device or a dedicated application interface. The computing device herein may be for example a desktop, a portable, a smart phone, a tablet and the like.

The statement analyzer 12 is configured to analyze the descriptive statement currently obtained by the interface 11, so as to extract predefined class information. The statement analyzer 12 may perform statement analysis using the natural language processing technology. The statement analyzer 12 may be implemented by means of a statement recognition model trained in advance based on a large amount of corpus data.

The querier 13 is configured to determine whether a query condition(s) is met based on the predefined class information extracted by the statement analyzer 12. If yes, it queries to obtain triage information and informs the user of the triage information obtained by querying through the interface 11, for example, displaying the triage information to the user in a dialogue box. The querier 13 for example may include a medical knowledge base and a diagnosis algorithm module. The medical knowledge base for example is a pre-constructed database including groups of related class information for various diseases. The diagnosis algorithm module includes e.g., a decision tree built by clinicians based on their experiences. When performing query, the querier 13 may perform query to the groups of information corresponding to diseases according to every piece of predefined class information recognized from the descriptive statement. When the query result includes a plurality of diseases, the probability of predefined class information combination identifying a disease may be determined based on correlations between every piece of predefined class information and the disease. One or more diseases with the highest probability are selected for determining the triage information.

In some embodiments, the intelligent triage system may further comprise an inquiry module 14. The inquiry module may be configured to generate an inquiry statement, when the statement analyzer 12 confirms that the currently obtained descriptive statement does not include any predefined class information, or when the querier 13 determined that the query condition is not met. The generated inquiry statement is displayed to the user through the interface 11, for example, displayed in the dialogue box of a user interface, so that more descriptive statements containing disease related information inputted by the user may be obtained.

In some embodiments, the intelligent triage system may further comprise a remote manual diagnosis module 15. The remote manual diagnosis module may be configured to obtain a descriptive statement through the interface 11, so as to perform manual triage, and transmit remote manual diagnosis information to the interface 11 so as to communicate it to the user. In some examples, the remote manual diagnosis module 15 may further cause the inquiry information to be displayed to the user through the interface 11. In an embodiment of this disclosure, the querier 13 may be configured to, when it determines based on the predefined class information that the query condition is not met and determines that the number of times of obtaining the descriptive statements via the inquiry module 14 exceeds a threshold, instruct the interface 11 to establish a connection with the remote manual diagnosis module 15, so as to provide manual triage consulting service by remote professionals.

In some embodiments, the intelligent triage system according to an embodiment of this disclosure may further comprise a registration module 16. The registration module may be configured to provide a link associated with the triage information in for example the dialogue box through the interface 11, so as to help the user to accomplish the registration operation. The link may be a hyperlink to a registration interface.

It should be understood that for the sake of clarity, the embodiments of the present invention have been described with reference to different functional modules. However, it is obvious that any appropriate allocation of the functionalities between different functional modules may be used without deviating from the present invention. Therefore, reference to a particular functional module is only regarded as reference to the appropriate means for providing the described functionality, rather than indicating strict logic or physical structure or organization.

Generally speaking, the terms “module” and “function” used herein may be implemented using hardware elements, software elements, or a combination of both. Examples of hardware elements may include devices, components, processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. In the case of software implementation, the module, function or logic represents program codes for executing designated tasks when being on a processor (e.g., one or more CPUs) or being executed by the processor. The program codes may be stored in one or more computer readable storage devices.

Various aspects of the present invention may be used separately, in combination or in various arrangements not specifically discussed in the preceding embodiments, therefore, the application thereof is not limited to the details and arrangements of the components expounded in the above specification or shown in the figures. For example, some aspects described in one embodiment may be combined with some aspects described in other embodiments in any way.

What are stated above are exemplary embodiments of this disclosure, rather than limitations to the protection scope of this disclosure. This disclosure is not limited to the above embodiments. Any modifications, equivalent replacements and improvements made within the spirit and the principle of the invention shall all fall into the protection scope of the invention.

1. A method for performing triage implemented by a computing device, comprising:
obtaining a descriptive statement inputted by a user via the computing device;
analyzing the descriptive statement to extract predefined class information, the predefined class information being disease related information that belongs to a predefined class;
querying, according to the predefined class information, to obtain the triage information when the query condition for querying triage information is met based on the predefined class information, and informing the user of the triage information.

2. The method as claimed in claim 1, wherein, when it fails to extract the predefined class information from the descriptive statement, or when it is determined based on the predefined class information that the query condition is not met, an inquiry statement is generated and displayed, so as to obtain a next descriptive statement inputted by the user.

3. The method as claimed in claim 1, wherein the analyzing the descriptive statement to extract predefined class information comprises:
recognizing a named entity in the descriptive statement through a pre-trained named entity recognition model.

4. The method as claimed in claim 3, further comprising:
extracting the recognized named entity as the predefined class information when the recognized named entity is a predefined class named entity.

5. The method as claimed in claim 1, wherein the query condition includes at least one of the following:
the extracted predefined class information includes particular class information;
the extracted predefined class information includes a predetermined amount of particular class information; and
the extracted predefined class information includes a piece of particular class information and the piece of particular class information is not included in a set of common predefined class information corresponding to a plurality of diseases.

6. The method as claimed in claim 1, wherein the querying to obtain the triage information comprises:
classifying each of pieces of extracted predefined class information through a pre-trained classification model; and
querying, at least according to the pieces of extracted predefined class information and their respective classes, to obtain the triage information.

7. The method as claimed in claim 1, wherein the querying to obtain the triage information comprises:
querying for triage information according to a pre-constructed decision tree, at least based on symptom information in the extracted predefined class information.

8. The method as claimed in claim 1, wherein the informing the user of the triage information comprises displaying the triage information on a user interface of the computing device, and when it is determined based on the predefined class information that the query condition is not met, the user interface is jumped to a remote manual diagnosis interface when the number of times of obtaining the descriptive statement exceeds a threshold.

9. The method as claimed in claim 1, wherein the method further comprises:
generating and displaying a statement that inquires whether to register or not after the triage information has been given; and
determining whether to provide a link associated with the triage information based on response information inputted by the user.

10. The method as claimed in claim 1, wherein the descriptive statement is analyzed through natural language processing (NLP).

11. The method as claimed in claim 1, wherein the predefined class information includes any one or more of symptom class information, physiological parameter class information, time class information, basic feature class information, and medical history class information.

12. The method as claimed in claim 1, further comprising:
calculating a total weighted value of the extracted predefined class information after the extracting the predefined class information, and
determining that the query condition is met when the weighted value exceeds a predefined threshold.

13. The method as claimed in claim 1, wherein the triage information includes at least one of disease information and corresponding department information.

14. A system for triage implemented by a computing device, comprising:
an interface configured to obtain a descriptive statement inputted by a user;
a statement analyzer configured to analyze the descriptive statement to extract predefined class information, the predefined class information being disease related information that belongs to a predefined class; and
a querier configured to query, according to the predefined class information, to obtain the triage information, when a query condition for querying triage information is met based on the predefined class information;
wherein the interface is further configured to inform the user of the triage information.

15. The system as claimed in claim 14, wherein the query condition includes at least one of the following:
the extracted predefined class information includes particular class information;
the extracted predefined class information includes a predetermined amount of particular class information; and
the extracted predefined class information includes a piece of particular class information and the piece of particular class information is not included in a set of common predefined class information corresponding to a plurality of diseases.

16. The system as claimed in claim 14, further comprising:
an inquiry module configured to generate an inquiry statement when the statement analyzer fails to extract the predefined class information from the currently obtained descriptive statement, or when the querier determines that the query condition is not met,
wherein the interface is further configured to display the inquiry statement so as to obtain a next descriptive statement inputted by the user.

17. The system as claimed in claim 14, further comprising:
a remote manual diagnosis module configured to obtain the descriptive statement through the interface so as to perform manual triage, and send remote manual triage information to the interface for display,
wherein the querier is further configured to, when it is determined based on the predefined class information that the query condition is not met, instruct the interface to establish a connection with the remote manual diagnosis module to perform manual triage when the number of times of obtaining the descriptive statement has exceeded a threshold.

18. The system as claimed in claim 14, further comprising:
a registration module configured to generate a link associated with the triage information,
wherein the interface is further configured to cause the link to be displayed to the user.

19. A computing device, comprising a memory, a processor and a computer program stored on the memory and running on the processor, wherein the processor, when executing the program, performs the steps of the method according to claim 1.

20. A computer readable storage medium, on which a computer readable instruction is stored, wherein the instruction, when being executed by the processor, implements the steps of the method according to claim 1.
